(19) United States
(12) Patent Application Publication
(10) Pub. No.: US 2024/0082138 A1
(43) Pub. Date: Mar. 14, 2024

(54) ACIDIC GLUCOSE-BASED HAIR CARE COMPOSITION

(71) Applicants: Paul R. Chambers, JR., Chicago, IL (US); Angela Adams McGhee, Conway, AR (US)

(72) Inventors: Paul R. Chambers, JR., Chicago, IL (US); Angela Adams McGhee, Conway, AR (US)

(21) Appl. No.: 18/323,747

(22) Filed: May 25, 2023

Related U.S. Application Data
(60) Provisional application No. 63/375,437, filed on Sep. 13, 2022.

(51) Int. Cl. A61K 8/73; A61K 8/41; A61Q 5/00

(52) U.S. Cl. CPC A61K 8/73; A61K 8/416; A61Q 5/00; A61K 2800/43; A61K 2800/522; A61K 2800/524

(57) ABSTRACT

An aqueous acidic haircare composition includes a glucose-based conditioning polymer; a humectant; a moisturizer; a surfactant; an antioxidant; and a preservative. The composition has a pH of about 3.5 to about 4.7. A method of treating damaged hair comprises rinsing the hair with tepid water and applying the composition. The composition neutralizes the hair shaft, reduces swelling of the cuticle, replenishes and protects the core polypeptide chain, strengthens existing disulfide bonds, and creates new ionic and hydrogen bonds within the hair structure. It makes coarse, afro-textured hair, curly hair, and wavy hair easier to manage.

### CROSS-REFERENCE TO RELATED APPLICATION

This application claims the benefit of priority of U.S. provisional application No. 63/375,437, filed Sep. 13, 2022, the contents of which are herein incorporated by reference.

### BACKGROUND OF THE INVENTION

The present invention relates to hair products and, more particularly, to an acidic glucose-based hair care composition.

Many people are plagued by hair breakage, dry scalp, thinning hair, and hard to manage and style hair. Damaged hair swells where the cuticle layers are exposed to stressors such as highly alkaline products, hot tools, and other chemicals that cause the cortex to be exposed to further damage. Historically, most solutions for damaged hair and dry scalp involved ingredients which act primarily at the surface of the hair, with limited penetration. These have included various forms of oils, waxes, hydrolyzed wheat proteins, and water-insoluble silicones. They inhibit moisture from penetrating throughout the hair strands, causing hair loss and constant dryness to hair and scalp. They do not address the internal structure of the hair strand, nor do they address the scalp in reducing inflammation due to heating implements as well as high alkalinity styling products and chemical services. Oils, waxes & silicones have molecular weights too large to penetrate past the cuticular layers in the hair that protect the cortex. Oils and proteins are also ineffective to adjust pH and reduce inflammation, which is one of the major reasons for damage to unruly hair and dry scalp.

Most currently available shampoos have a pH between about 6 and about 9.5; most currently available hair bleach compositions, hair color compositions, and perm corn positions have a pH between about 8.5 and 11; and most currently available sodium hydroxide-based “relaxers” have a pH of at least about 11. These prior art compositions cause the hair cuticle to swell, compromising the bonds and causing +linkages to be broken or damaged. As a result, the hair loses strength, elasticity, resiliency, and shine. Note that the pH scale is logarithmic so an increase of one from neutral pH 7 indicates a composition that is ten-fold more alkaline.

As can be seen, there is a need for a product that remedies damaged hair and dry scalp, reduces inflammation, penetrates the hair shaft to the protein chain, stops breakage, and restores moisture balance.

### SUMMARY OF THE INVENTION

In one aspect of the present invention, an aqueous acidic haircare composition comprises a glucose-based conditioning polymer; a humectant; a moisturizer; a surfactant; an antioxidant; and a preservative; wherein the acidic haircare composition has a pH of about 3.5 to about 4.7.

In another aspect of the present invention, a method of treating damaged hair comprises rinsing the hair with tepid water and applying the aqueous acidic haircare composition.

The inventive composition neutralizes the hair shaft, reduces swelling of the cuticle, replenishes and protects the core polypeptide chain, strengthens existing disulfide bonds, and creates new ionic and hydrogen bonds within the hair structure.

The composition described herein makes coarse, afro-textured hair, curly hair, and wavy hair easier to manage with natural moisture. The composition strengthens and nourishes all hair types, promoting hair growth and dramatically reversing damage, while it alleviates inflammation in the hair and scalp. A single usage of the product alleviates inflammation in the hair and scalp, making hair fuller, reducing thinning hair, reducing hair loss, and reducing dry itchy scalp.

These and other features, aspects and advantages of the present invention will become better understood with reference to the following drawings, description, and claims.

### BRIEF DESCRIPTION OF THE DRAWINGS

FIG. 1 is a schematic flowchart of a process according to an embodiment of the present invention;

FIG. 2 is a photomicrograph of damaged hair; and

FIG. 3 is another photomicrograph thereof, shown after treatment with the inventive composition.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is an acidic glucose-based haircare system that reduces inflammation and restores strength and moisture for all hair types.

The inventive hair care composition is sometimes referred to herein as “PBond Hair Care” or “PBond” treatment.

The core elements within the formula are water-soluble ingredients. Starting with the acidic glucose-based water bond complex, water soluble botanicals, humectants, and co-emulsifiers were carefully layered to maintain sustainable moisture in the scalp and hair. Providing specific concentrated levels of each ingredient facilitates fast absorption and penetration deep into the hair structure inside the cortex and across the polypeptide chain to dramatically reduce inflammation, provide sustainable moisture balance, and recreate new bonds for improved manageability for all hair types.

The core elements within the formula must be water soluble and acidic, including the glucose-based water bond complex, botanicals, humectants, and co-emulsifiers to maintain moisture in the scalp and hair. The order of addition may be changed provided that the concentration levels of each ingredient are generally unchanged.

The pH of the composition is generally acidic, e.g., it may be in the range of about 4.2 to about 4.7. In some embodiments, the pH may be adjusted slightly under 4.0 to combat escalated inflammation in the hair and scalp. For example, the pH may be as low as about 3.5. In these very low pH embodiments, a heated dryer is unnecessary for the product to work at optimum levels.

Hydroxypropyl gluconamide and hydroxypropyl ammonium gluconate are water-soluble molecules, derived from glucose, which facilitate absorption and penetration deep into the hair structure inside the cortex and across the polypeptide chain to dramatically strengthen and moisturize the hair, creating new hydrogen bonds and ionic bonds and reconnecting and strengthening weak bonds. These components are effective in creating new hydrogen bonds and ionic bonds within the polypeptide protein chain of the hair shaft.

In addition, water-soluble extracts and co-solvent moisturizing ingredients are combined in an aqueous solution to maximize moisture levels/hydration and nutrition on a cellular level to the scalp and hair. As a result, the inventive composition may be used to address skin/scalp disorders. The composition provides a continuous moisturizing umbrella that extends long after the first usage of the product. The inventive composition “layers” humectants and botanical extracts to provide instantaneous absorption and penetration into the hair to alleviate buildup, heaviness, and stickiness. As a result, the hair follicle and shaft are uninhibited so the strand may move, sustain shine, and grow!

The water-based composition starts with an acidic water bond base complex. This is the foundation for strengthening recreation of broken bonds and elasticity within the inner structure of the hair. It is augmented by 100% Certifiable water soluble organic botanicals and humectants that interact consistently with other ingredients for long term results in combatting dryness, breakage, and hair thinning. Individually, each ingredient is selected to be acidic & water soluble to interact in the layering process of acidic glucose-based complex chemistry in consistently rebuilding, restoring, and sustaining the inner structure of the hair & scalp.

In addition to an acidic glucose water bond complex, we layered plant based, organic, water-soluble humectants and co-emulsifiers to help address issues within the scalp, reversing hair loss, thinning, and dehydration. For example, the water-soluble organic botanical humectants may include Aloe Barbadensis, Panthenol, Bamboo extract, and Nettle leaf extract to provide medicinal and moisture enhancing properties, protecting hair and scalp in various climates and weather conditions. The additional ingredients may include those discussed below.

Aloe Vera (Aloe Barbadensis) and propanediol boost hydration, reduce itchy scalp, and strengthen hair while enabling more absorption into the hair and scalp by maintaining a consistent viscosity within the composition.

Panthenol (a moisturizer and lubricating compound) and Polysorbate 80 (a surfactant and emulsifier) may be used in some embodiments to enhance the skin and hair texture, increase hair body, and promote hair growth and sheen.

Cetrimonium Chloride is a quaternary ammonium compound used as a preservative in some embodiments. It serves as an excellent conditioning agent for hair, with anti-static, anti-frizz, and detangling properties. It also functions as an emulsifier to stabilize emulsions and as a surfactant, boosting rinse-off.

In some embodiments, a sodium salt of pyroglutamic acid (Sodium pyroglutamic acid [PCA]) may be used as a conditioning agent.

Phenoxyethanol is a preservative that may be included to protect the water-based formulation from a broad-spectrum of pathogenic activity.

Caprylyl glycol is water-soluble and is used as a skin conditioning agent that naturally moisturizes scalp and hair.

Bambusa Vulgaris (Bamboo Extract) improves strength and moisture retention.

Urtica Dioica (Organic Nettle Extract) reduces itchy scalp & dandruff, increases longevity of shine, and provides cuticular strength for consistent moisture balance. For example, the ingredient may be an extract from Urtica dioica leaves.

Sorbic Acid may be added to prohibit yeast & mold to preserve the longevity of the water-based product, while maintaining its effectiveness. In particular, sorbic acid may be used to preserve Bambusa Vulgaris (Bamboo Extract) and Urtica Dioica (Organic Nettle Extract), organic ingredients that may be used to improve strength, smoothness, and shine and to reduce dandruff.

In some embodiments, the inventive composition may exclude one or more ingredients selected from the group consisting of hydrolyzed wheat proteins, mineral oils, coconut oils, castor oils, argan oils, water-insoluble silicones, and combinations thereof. In other words, the excluded ingredients are absent from the hair composition.

This unique formula relies on glucose-based chemistry and may be used in an acidic water-based hair care line including haircare components selected from the group consisting of shampoos, treatments such as leave-in conditioner, and styling and finishing products such as hydrating and styling gel and conditioner, e.g., a styling curl gel. The inventive composition is lightweight and penetrating so it quickly absorbs into the hair and scalp to strengthen, moisturize, and hydrate all hair types, including afro-texture hair. The inventive vegan, humectant glucose-based composition advantageously provides long term solutions in combating dryness, swelling, and buildup on the scalp and hair by creating new bonds inside the hair shaft, providing strength and manageability to the hair for easier styling and sustainability.

In some embodiments, the composition may include additional ingredients selected from the group consisting of Sodium C14-16 Sulfonate, Cocamidopropyl Betaine, Distearoylethyl Dimonium Chloride, Stearamidopropyl Dimethylamine, Sodium Bicarbonate, Rosa bamascena (Rose) Flower Distillate, Ceteareth-20, Glyceryl Distearate, Sclerocarya birrea (Organic Marula Oil), Vitis vinifera (Grapeseed Oil), Sorbitan Oleate, Palm Oil, D-alpha Tocopheryl Acetate, Glycine soja (Soybean Oil), Biotin, Cetearyl alcohol, Glycerin, Hydrogenated Ethylhexyl Olivate, Polyquaternium, and any combination thereof.

In a method of use according to an embodiment of the present invention, the hair care composition may be applied to hair that is badly breaking or shedding after rinsing the hair with tepid water. After applying the composition as a mist, the stylist may place a plastic cap over the hair and heat the covered hair for a predetermined period, such as 10 minutes. Inflammation may be drastically reduced as the hair is returned to an acidic pH balance, stopping breakage and shedding. In a method of use according to another embodiment of the present invention, the hair care composition may be applied with permanent relaxers on afro textured hair. The treatment is both a neutralizer and strengthening treatment infused with moisture and hydrating properties to bring hair back to its healthiest condition. The “PBond” treatment replaces previously commercially available neutralizing shampoo and conditioner, normally used after rinsing relaxer out of relaxed hair, which do little to correct the damage that alkaline chemicals cause. The “PBond” treatment creates new bonds to replace bonds that have been permanently destroyed during the relaxer application. It restores weakened bonds, provides nutrition to the scalp and hair, and dramatically reduces inflammation (i.e., swelling of the cuticle strand) and porosity induced by the high alkalinity of the relaxer (generally about 12-14). The hair is hydrated and quickly recovers its moisture balance for improved elasticity, softness, and natural shine without the aid of silicones, oils, or waxes. After application of the “PBond” treatment, the hair is ready for styling.

In a method of use according to an embodiment of the present invention, the composition may be mixed with high alkaline bleach, used for highlights, or with color to maintain the integrity of the hair and eliminate brassiness that otherwise occur due to the application of the bleach and color. It also improves color retention through realignment reduction in porosity. One may apply several mists of the hair care composition to a container of color or bleach.

In a method of use according to yet another embodiment of the present invention, the hair care composition may be applied to dry hair before applying color or bleach. The bleach applied to the hair weakens bonds; the inventive composition is believed to create new bonds, making the hair stronger.

In a method of use according to another embodiment of the present invention, the hair care composition may be used when rinsing hair after a relaxer. The stylist may mist the composition onto the entire head of hair from scalp to ends. The application may be left on the hair for about 10 minutes. In some cases, depending on hair density, texture, and condition, the stylist may place a plastic cap over the hair and heat the covered hair under a medium hair dryer for a predetermined period, such as about 10 minutes, to enhance the strength of the treatment. The stylist may then rinse and style the hair.

### EXAMPLE

A formulation comprising the following components was prepared by blending the non-ionic surfactant, fragrance, and preservative to make a first blend and adding the first blend to a second blend of the remaining ingredients.

| | | Range, |
|---|---|---|
| Component | Ingredient (source) | weight % |
| External analgesic, | Aloe vera leaf, powdered or liquid (Bulk | 2-8 |
| humectant | Apothecary ™) | |
| Antioxidant | Bamboo extract powder (Bulk | 0.5-4 |
| | Supplements ™) | |
| Conditioning polymer | Hydroxypropyl gluconamide and | 2-7 |
| | Hydroxypropyl ammonium gluconate (sold | |
| | as FiberHance ™ BM Solution, | |
| | Chempoint ®) | |
| Surfactant - emulsifying | cetrimonium chloride (30%, Naturally | 0.5-1.5 |
| agent | thinking) | |
| Moisturizer, lubricant | Panthenol (Organic Creations) | 0.5-3 |
| Moisturizer, emollient | 1,3-propanediol (NatureSilk, Ingredients | 1-5 |
| | TO DIE FOR ™) | |
| Humectant | sodium PCA (50%, Lotioncrafter ®) | 1-4 |
| Non-ionic surfactant | polysorbate 80 | 2-8 |
| Fragrance | Fragrance oil (Wellington Fragrance Co.) | 0.2-1 |
| Preservative | Kraftiphen plus (Kraft Chemical ® Co. | 0-3 |
| Solvent | water | Balance |
| | | to 100% |

The inventive composition neutralizes the hair shaft, reduces swelling of the cuticle, replenishes, and protects the core polypeptide chain, strengthens existing disulfide bonds, and creates new ionic and hydrogen bonds within the hair structure.

The hair exhibits even porosity, body, and movement. These properties were restored back to almost virgin hair conditions.

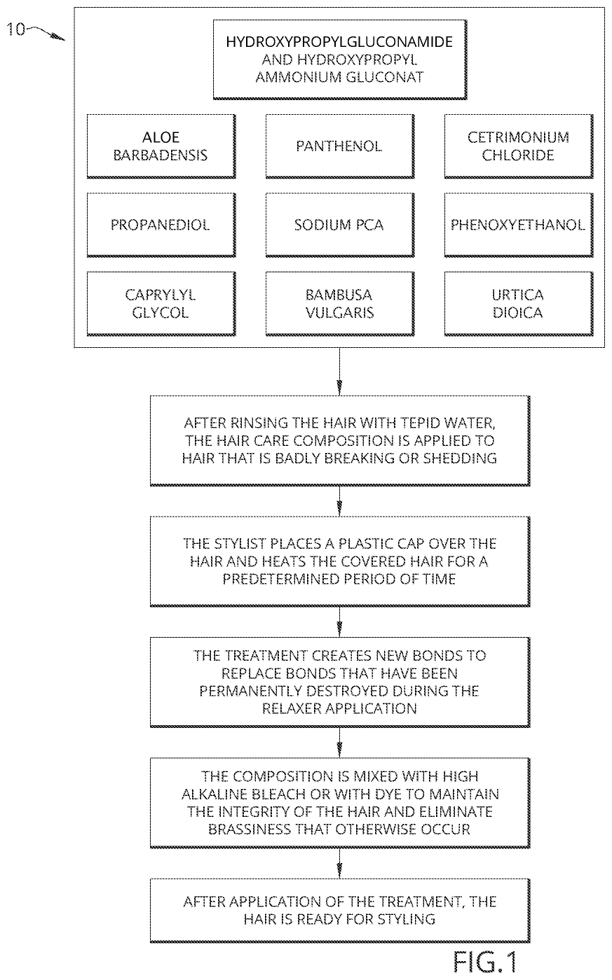

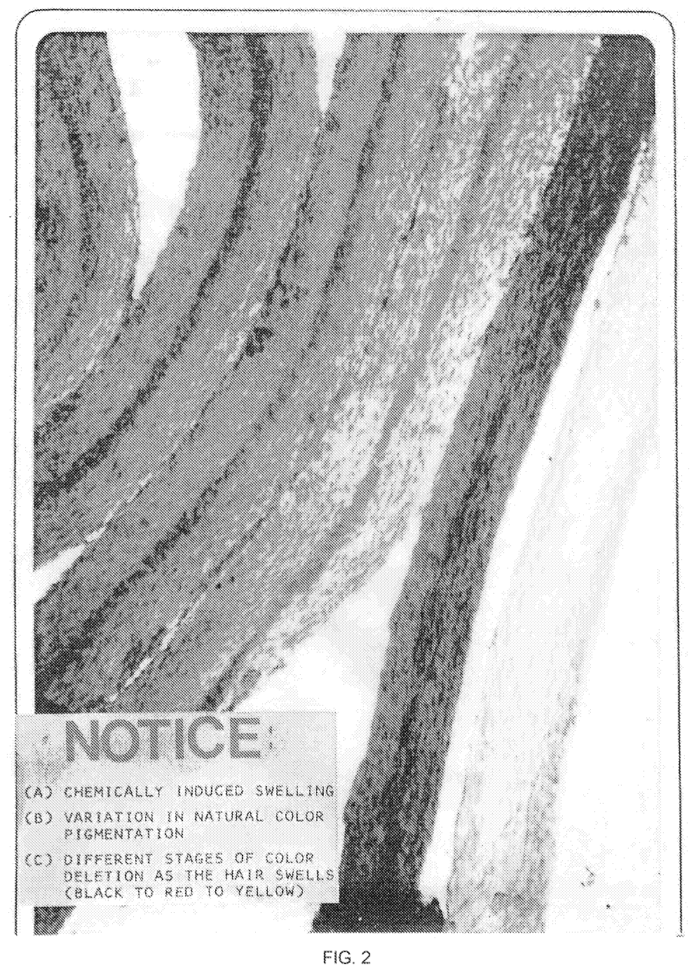

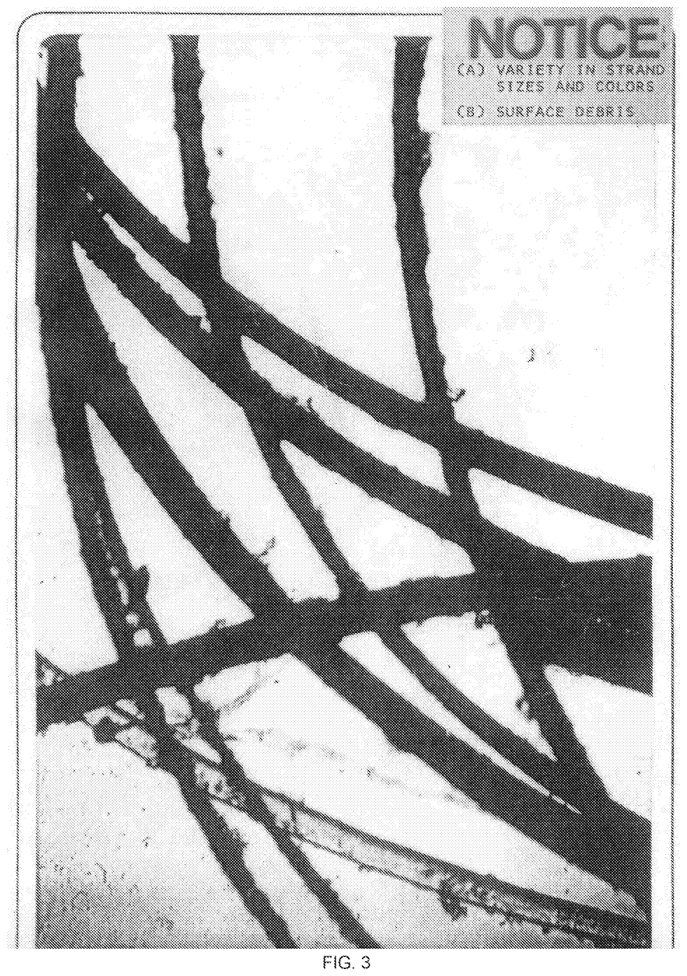

Referring to FIGS. 1 through 3, FIG. 1 illustrates a method of treating hair with a composition 10 according to an embodiment of the present invention. The exemplary composition 10 comprises hydroxypropylgluconamide and hydroxypropyl ammonium gluconate; aloe barbadensis; panthenol; cetrimonium chloride; propanediol; sodium PCA; phenoxyethanol; caprylyl glycol; Bambusa vulgaris; and Urtica dioica. After the hair is rinsed in tepid water, the composition 10 is applied to the hair. A plastic cap is placed over the hair and the covered hair is heated. Alternatively, or in addition, the composition 10 is mixed with a bleach composition or mixed with a hair dye composition and applied to hair. The treatment creates new bonds to replace bonds that have been permanently destroyed during relaxer application. After application of the composition 10, with or without application of heat, the hair may be styled. FIG. 2 shows damaged hair before treatment; note the swelling and discoloration. FIG. 3 shows the same strands of hair after treatment with composition 10.

It should be understood, of course, that the foregoing relates to exemplary embodiments of the invention and that modifications may be made without departing from the spirit and scope of the invention as set forth in the following claims.

What is claimed is:

1. An aqueous acidic haircare composition, comprising
a glucose-based conditioning polymer;
a humectant;
a moisturizer;
a surfactant;
an antioxidant; and
a preservative;
wherein the aqueous acidic haircare composition has a pH of about 3.5 to about 4.7.

2. The aqueous acidic haircare composition of claim 1, wherein the glucose-based conditioning polymer, the humectant, the surfactant, the antioxidant, and the preservative are each present in an amount of from about 0.5 to about 8 weight %.

3. The aqueous acidic haircare composition of claim 1, wherein the glucose-based conditioning polymer is a blend of hydroxypropylammonium gluconate and hydroxypropyl gluconamide.

4. The aqueous acidic haircare composition of claim 1, wherein the humectant is selected from the group consisting of aloe barbadensis, panthenol, Bambusa vulgaris extract, Urtica Dioica leaf extract, propanediol, and any combination thereof.

5. The aqueous acidic haircare composition of claim 1, wherein the moisturizer is selected from the group consisting of caprylyl glycol, Bambusa vulgaris, panthenol, propanediol, and any combination thereof.

6. The aqueous acidic haircare composition of claim 1, wherein the surfactant is selected from the group consisting of a quaternary ammonium compound, a polysorbate, and any combination thereof.

7. The aqueous acidic haircare composition of claim 1, wherein the antioxidant is Bambusa vulgaris extract.

8. The aqueous acidic haircare composition of claim 1, wherein the preservative is selected from the group consisting of a quaternary ammonium compound, phenoxyethanol, sorbic acid, and any combination thereof.

9. The aqueous acidic haircare composition of claim 1, further comprising a sodium salt of pyroglutamic acid.

10. The aqueous acidic haircare composition of claim 1, further comprising caprylyl glycol.

11. The aqueous acidic haircare composition of claim 1, excluding ingredients selected from the group consisting of hydrolyzed wheat proteins, mineral oils, coconut oils, castor oils, argan oils, water-insoluble silicones, and combinations thereof.

12. The aqueous acidic haircare composition of claim 1, further comprising a haircare component selected from the group consisting of: shampoo, conditioner, leave-in conditioner, styling gel, hydrating oil, and any combination thereof.

13. A method of treating damaged hair, comprising:
rinsing the hair with tepid water; and
applying the aqueous acidic haircare composition of claim 1 to the hair.

14. The method of claim 13, further comprising:
placing a plastic cap over the hair; and
heating the plastic cap covered hair for a predetermined period.

15. The method of claim 13, wherein the aqueous acidic haircare composition is applied as a mist onto an entire head of hair from scalp to ends.

16. The method of claim 14, wherein the predetermined period is about 10 minutes.

17. The method of claim 13, wherein the aqueous acidic haircare composition is applied with a permanent relaxer.

18. The method of claim 13, wherein the aqueous acidic haircare composition is applied mixed with high alkaline bleach.

19. The method of claim 13, wherein the aqueous acidic haircare composition is applied with hair dye.
